(19) United States
(12) Patent Application Publication
(10) Pub. No.: US 2008/0020004 A1
(43) Pub. Date: Jan. 24, 2008

(54) Hair-Treatment Agent Comprising Terpolymer Of Vinylpyrrolidone, Methacrylamide And Vinylimidazole And Active Ingredients And Additives

(76) Inventors: Susanne Birkel, Darmstadt (DE); Thomas Krause, Darmstadt (DE); Michael Franzke, Rossdorf (DE)

Correspondence Address: MICHAEL J. STRIKER, 103 EAST NECK ROAD, HUNTINGTON, NY, 11743, US

(21) Appl. No.: 10/589,372

(22) PCT Filed: Feb. 18, 2005
(86) PCT No.: PCT/EP05/01693

§ 371 Date: May 4, 2007

(30) Foreign Application Priority Data
Feb. 18, 2004 (DE) 202004002471.3

(51) Int. Cl. A61K 8/81; A61K 8/06; A61Q 5/06

(52) U.S. Cl. 424/401; 424/70.12; 424/70.15

(57) ABSTRACT

A hair-treatment agent is described with a content of a combination of a terpolymer constructed from vinylpyrrolidone, methacrylamide and vinylimidazole, and at least one active ingredient or additive chosen from viscosity-modifying substances, haircare substances, hair-setting substances, silicone compounds, photoprotective substances, oils, waxes, preservatives, pigments, soluble dyes, particulate substances, and surfactants in a suitable cosmetic base. The hair-treatment agent can be in the form of a gel, a viscous lotion, a spray gel, an O/W emulsion, a W/O emulsion, a microemulsion, a hair spray, a foamable product in combination with a device for foaming, or in the form of a hair wax. Preference is given to products which are foamable with a propellant in which the active ingredient or additive is chosen from chitosan, chitosan salts, chitosan derivatives, cationic cellulose compounds and copolymers of vinylpyrrolidone, vinylcaprolactam and a basic acrylamide monomer.

The invention relates to a hair-treatment agent with a content of a special terpolymer of vinylpyrrolidone, methacrylamide and vinylimidazole in combination with selected active ingredients and/or additives.

In order to set and give hold to human hair in order to stabilize a created hairstyle, in order to improve the styling properties or the hair structure, hair-treatment agents in the form of setting lotions, aerosol and nonaerosol sprays, setting foams, gels etc. are used. The cosmetic, hair-setting or haircare polymers usually used for these purposes exhibit good setting and/or care properties in aqueous, alcoholic or aqueous-alcoholic media. However, not all requirements such as, for example, feel, shine, combability, durability of setting etc. are often still not satisfied in an entirely satisfactory manner. WO 03/092640 discloses water-soluble copolymers with (meth)acrylamide units and their use in hair-treatment agents. SÖFW-Journal, 12-2003, page 65-72 describes hairstyling products which comprise a vinylpyrrolidone/methacrylamide/vinylimidazole copolymer. It was the object to further improve the film-forming, hair-setting, haircare and/or application properties of polymer-containing preparations.

It has now been found that the object is achieved by a combination of
(A) a terpolymer constructed from vinylpyrrolidone, methacrylamide and vinylimidazole and
(B) at least-one active ingredient or additive chosen from viscosity-modifying substances, haircare substances, hair-setting substances, silicone compounds, photoprotective substances, oils, waxes, preservatives, pigments, soluble dyes,
   particulate substances, and surfactants in a suitable cosmetic base.

The terpolymer (A) is present in the agent according to the invention preferably in an amount of from 0.01 to 20% by weight, particularly preferably from 0.1 to 10% by weight, very particularly preferably from 0.5 to 5% by weight, and the additive (B) is present in an amount of from 0.01 to 20% by weight, particularly preferably from 0.05 to 10% by weight, very particularly preferably from 0.1 to 5% by weight.

Suitable terpolymers (A) are those which can be prepared from the free-radically polymerizable monomers vinylpyrrolidone, methacrylamide and vinylimidazole. The preparation of such a polymer is described in WO 03/092640, e.g. example Nos. 61, 62, 64 and 65, and is commercially available under the name Luviflex® Clear (BASF) (INCI name: VP/Methacrylamide/Vinyl Imidazole Copolymer).

In one embodiment, the agent according to the invention comprises at least one viscosity-modifying substance in an amount of from preferably 0.01 to 20% by weight or 0.05 to 10% by weight or particularly preferably from 0.1 to 5% by weight. The viscosity-modifying substance is preferably a thickening polymer, chosen from copolymers of at least one first monomer type, which is chosen from acrylic acid and methacrylic acid, and at least one second monomer type, which is chosen from esters of acrylic acid and ethoxylated fatty alcohol; crosslinked polyacrylic acid; crosslinked copolymers of at least one first monomer type, which is chosen from acrylic acid and methacrylic acid, and at least one second monomer type, which is chosen from esters of acrylic acid with C10- to C30-alcohols; copolymers of at least one first monomer type, which is chosen from acrylic acid and methacrylic acid, and at least one second monomer type, which is chosen from esters of itaconic acid and ethoxylated fatty alcohol; copolymers of at least one first monomer type, which is chosen from acrylic acid and methacrylic acid, at least one second monomer type, which is chosen from esters of itaconic acid and ethoxylated C10- to C30-alcohol and a third monomer type, chosen from C1- to C4-aminoalkyl acrylates; copolymers of two or more monomers chosen from acrylic acid, methacrylic acid, acrylic esters and methacrylic esters; copolymers of vinylpyrrolidone and ammonium acryloyldimethyltaurate; copolymers of ammonium acryloyldimethyltaurate and monomers chosen from esters of methacrylic acid and ethoxylated fatty alcohols; hydroxyethylcellulose; hydroxypropylcellulose; hydroxypropylguar; glyceryl polyacrylate; glyceryl polymethacrylate; copolymers of at least one C2-, C3- or C4-alkylene and styrene; polyurethanes; hydroxypropyl starch phosphate; polyacrylamide; copolymer of maleic anhydride and methyl vinyl ether crosslinked with decadiene; carob seed flour; guar gum; xanthan; dehydroxanthan; carrageenan; karaya gum; hydrolyzed corn starch; copolymers of polyethylene oxide, fatty alcohols and saturated methylenediphenyl diisocyanate (e.g. PEG-150/stearyl alcohol/SMDI copolymer).

In one embodiment, the agent according to the invention comprises, as haircare or hair-setting additive, at least one polymer with anionic or anionizable groups in an amount of from preferably 0.01 to 20% by weight or from 0.05 to 10% by weight, particularly preferably from 0.1 to 5% by weight. Anionizable groups are understood as meaning acid groups, such as, for example, carboxylic acid, sulfonic acid or phosphoric acid groups, which can be deprotonated by means of customary bases, such as, for example, organic amines or alkali metal or alkaline earth metal hydroxides.

The polymers of component (B) can be partially or completely neutralized with a basic neutralizing agent. Preference is given to those agents in which, in the polymer of component (B), the acid groups are neutralized to 50 to 100%, particularly preferably to 70-100%. Neutralizing agents which can be used are organic or inorganic bases. Examples of bases are, in particular, aminoalkanols, such as, for example, aminomethylpropanol (AMP), triethanolamine or monoethanolamine, but also ammonia, NaOH, KOH etc.

The polymer (B) may be a homopolymer or copolymer with natural-based or synthetic-based monomer units containing acid groups which is optionally copolymerized with comonomers which contain no acid groups. Suitable acid groups are sulfonic acid groups, phosphoric acid groups and carboxylic acid groups, of which the carboxylic acid groups are preferred. Suitable monomers containing acid groups are, for example, acrylic acid, methacrylic acid, crotonic acid, maleic acid or maleic anhydride, maleic monoesters, in particular the mono-C1-C7-alkyl esters of maleic acid, and aldehydocarboxylic acids or ketocarboxylic acids. Comonomers not substituted by acid groups are, for example, acrylamide, methacrylamide, alkyl- and dialkylacrylamide, alkyl- and dialkylmethacrylamide, alkyl acrylate, alkyl methacrylate, vinylcaprolactone, vinylpyrrolidone, vinyl ester, vinyl alcohol, propylene glycol or ethylene glycol, amine-substituted vinyl monomers such as, for example, dialkylaminoalkyl acrylate, dialkylaminoalkyl methacrylate, monoalkylaminoalkyl acrylate and monoalkylaminoalkyl methacrylate, where the alkyl groups of these monomers are preferably C1- to C7-alkyl groups, particularly preferably C1- to C3-alkyl groups.

Suitable polymers with acid groups are, in particular, homopolymers of acrylic acid or of methacrylic acid which are uncrosslinked or crosslinked with polyfunctional agents, copolymers of acrylic acid or methacrylic acid with monomers chosen from acrylic or methacrylic esters, acrylamides, methacrylamides and vinylpyrrolidone, homopolymers of crotonic acid, and copolymers of crotonic acid with monomers chosen from vinyl esters, acrylic or methacrylic esters, acrylamides and methacrylamides. A suitable natural polymer is, for example, Shellac.

Preferred polymers with acid groups are:

Terpolymers of acrylic acid, alkyl acrylate and N-alkylacrylamide (INCI name: Acrylates/Acrylamide Copolymer), in particular terpolymers of acrylic acid, ethyl acrylate and N-tert-butylacrylamide; crosslinked or uncrosslinked vinyl acetate/crotonic acid copolymers (INCI name: VA/Crotonates Copolymer); copolymers of one or more C1-C5-alkyl acrylates, in particular C2-C4-alkyl acrylates and at least one monomer chosen from acrylic acid or methacrylic acid (INCI name: Acrylates Copolymer), e.g. terpolymers of tert-butyl acrylate, ethyl acrylate and methacrylic acid; sodium polystyrenesulfonate; vinyl acetate/crotonic acid/vinyl alkanoate copolymers, e.g. copolymers of vinyl acetate, crotonic acid and vinyl propionate; copolymers of vinyl acetate, crotonic acid and vinyl neodecanoate (INCI names: VA/Crotonates/Vinyl Propionate Copolymer, VA/Crotonates/Vinyl Neodecanoate Copolymer); aminomethylpropanol acrylate copolymers; copolymers of vinylpyrrolidone and at least one further monomer chosen from acrylic acid and methacrylic acid, and optionally acrylic esters and methacrylic esters; copolymers of methyl vinyl ether and maleic monoalkyl esters (INCI names: Ethyl ester of PVM/MA Copolymer, Butyl ester of PVM/MA Copolymer); aminomethylpropanol salts of copolymers of allyl methacrylate and at least one further monomer chosen from acrylic acid, and methacrylic acid, and optionally acrylic esters and methacrylic esters; crosslinked copolymers of ethyl acrylate and methacrylic acid; copolymers of vinyl acetate, mono-n-butyl maleate and isobornyl acrylate; copolymers of two or more monomers chosen from acrylic acid and methacrylic acid, and optionally acrylic esters and methacrylic esters; copolymers of octylacrylamide and at least one monomer chosen from acrylic acid and methacrylic acid, and optionally acrylic esters and methacrylic esters; polyesters of diglycol, cyclohexanedimethanol, isophthalic acid and sulfoisophthalic acid, where the alkyl groups of the abovementioned polymers generally have preferably 1, 2, 3 or 4 carbon atoms.

In one embodiment, the agent according to the invention comprises, as haircare or hair-setting additive, at least one zwitterionic and/or amphoteric polymer in an amount of from preferably 0.01 to 20% by weight or from 0.05 to 10% by weight, particularly preferably from 0.1 to 5% by weight. Zwitterionic polymers at the same time have at least one anionic and at least one cationic charge. Amphoteric polymers have at least one acidic group (e.g. carboxylic acid or sulfonic acid group) and at least one basic group (e.g. amino group). Acid groups can be deprotonated by means of customary bases, such as, for example, organic amines or alkali metal or alkaline earth metal hydroxides.

Preferred zwitterionic or amphoteric polymers are:

Copolymers formed from alkylacrylamide, alkylaminoalkyl methacrylate and two or more monomers of acrylic acid and methacrylic acid, and optionally esters thereof, in particular copolymers of octylacrylamide, acrylic acid, butylaminoethyl methacrylate, methyl methacrylate and hydroxypropyl methacrylate (INCI name: octylacrylamide/Acrylates/Butylaminoethyl Methacrylate Copolymer); copolymers which are formed from at least one first monomer type which has quaternary amine groups, and at least one second monomer type which has acid groups; copolymers of fatty alcohol acrylates, of alkylamine oxide methacrylate and at least one monomer chosen from acrylic acid and methacrylic acid, and optionally acrylic esters and methacrylic esters, in particular copolymers of lauryl acrylate, stearyl acrylate, ethylamine oxide methacrylate and at least one monomer chosen from acrylic acid and methacrylic acid, and optionally esters thereof; copolymers of methacryloylethylbetaine and at least one monomer chosen from methacrylic acid and methacrylic esters; copolymers of acrylic acid, methyl acrylate and methacrylamidopropyltrimethylammonium chloride (INCI name: Polyquaternium-47); copolymers of acrylamidopropyltrimethylammonium chloride and acrylates or copolymers of acrylamide, acrylamidopropyltrimethylammonium chloride, 2-amidopropylacrylamidesulfonate and dimethylaminopropylamine (INCI name: Polyquaternium-43); oligomers or polymers, preparable from quaternary croton betaines or quaternary croton betaine esters.

In one embodiment, the agent according to the invention comprises, as haircare or hair-setting additive, at least one cationic polymer, i.e. a polymer with cationic or cationizable groups, in particular primary, secondary, tertiary or quaternary amine groups in an amount of from preferably 0.01 to 20% by weight or from 0.05 to 10% by weight, particularly preferably from 0.1 to 5% by weight. The cationic charge density is preferably 1 to 7 meq/g.

The suitable cation-active polymers are preferably hair-setting or hair-conditioning polymers. Suitable polymers of component (B) preferably comprise quaternary amine groups. The cationic polymers may be homopolymers or copolymers, where the quaternary nitrogen groups are present either in the polymer chain or preferably as substituent on one or more of the monomers. The monomers containing ammonium groups may be copolymerized with noncationic monomers. Suitable cationic monomers are unsaturated, free-radically polymerizable compounds which carry at least one cationic group, in particular ammonium-substituted vinyl monomers, such as, for example, trialkylmethacryloxyalkylammonium, trialkylacryloxyalkylammonium, dialkyldiallylammonium and quaternary vinylammonium monomers with cyclic, cationic nitrogen-containing groups, such as pyridinium, imidazolium or quaternary pyrrolidones, e.g. alkylvinylimidazolium, alkylvinylpyridinium, or alkylvinylpyrrolidone salts. The alkyl groups of these monomers are preferably lower alkyl groups, such as, for example, C1- to C7-alkyl groups, particularly preferably C1- to C3-alkyl groups.

The monomers containing ammonium groups may be copolymerized with noncationic monomers. Suitable comonomers are, for example, acrylamide, methacrylamide, alkyl- and dialkylacrylamide, alkyl- and dialkylmethacrylamide, alkyl acrylate, alkyl methacrylate, vinylcaprolactone, vinylcaprolactam, vinylpyrrolidone, vinyl esters, e.g. vinyl acetate, vinyl alcohol, propylene glycol or ethylene glycol, where the alkyl groups of these monomers are preferably C1- to C7-alkyl groups, particularly preferably C1- to C3-alkyl groups.

Suitable polymers with quaternary amine groups are, for example, the polymers described in the CTFA Cosmetic Ingredient Dictionary under the names Polyquaternium, such as methylvinylimidazolium chloride/vinylpyrrolidone copolymer (Polyquaternium-16) or quaternized vinylpyrrolidone/dimethylaminoethyl methacrylate copolymer (Polyquaternium-11), and quaternary silicone polymers and oligomers, such as, for example, silicone polymers with quaternary end groups (Quaternium-80).

Preferred synthetic-based cationic polymers:

Poly(dimethyldiallyammonium chloride); copolymers of acrylamide and dimethyldiallylammonium chloride; quaternary ammonium polymers formed by the reaction of diethyl sulfate and a copolymer of vinylpyrrolidone and dimethylaminoethyl methacrylate, in particular vinylpyrrolidone/dimethylaminoethyl methacrylate methosulfate copolymer (e.g. Gafquat® 755 N, Gafquat® 734); quaternary ammonium polymers of methylvinylimidazolium chloride and vinylpyrrolidone (e.g. LUVIQUAT® HM 550); Polyquaternium-35; Polyquaternium-57; polymer of trimethylammonium ethyl methacrylate chloride; terpolymers of dimethyldiallylammonium chloride, sodium acrylate and acrylamide (e.g. Merquat® Plus 3300); copolymers of vinylpyrrolidone, dimethylaminopropyl methacrylamide and methacryloylaminopropyllauryldimethylammonium chloride; terpolymers of vinylpyrrolidone, dimethylaminoethyl methacrylate and vinylcaprolactam (e.g. Gaffix® VC 713); vinylpyrrolidone/methacrylamidopropyltrimethylammonium chloride copolymers (e.g. Gafquat® HS 100); copolymers of vinylpyrrolidone and dimethylaminoethyl methacrylate; copolymers of vinylpyrrolidone, vinylcaprolactam and dimethylaminopropylacrylamide; polyesters or oligoesters constructed from at least one first monomer type which is chosen from hydroxy acid substituted by at least one quaternary ammonium group; dimethylpolysiloxanes terminally substituted by quaternary ammonium groups.

Preferred polymers with amino groups are copolymers of vinylpyrrolidone, vinylcaprolactam and a basic acrylamide monomer, where the basic acrylamide monomer is preferably chosen from dialkylaminoalkyl methacrylamide and dialkylaminoalkyl acrylamide, where the alkyl groups consist of 1 to 4 carbon atoms. Particular preference is given to dimethylaminopropyl methacrylamide. The preparation of such a polymer is described in WO 96/19971 and is commercially available under the name Aquaflex® SF 40 (ISP).

Suitable cationic polymers which are derived from natural polymers are, in particular, cationic derivatives of polysaccharides, for example cationic derivatives of cellulose, starch or guar. Also suitable are chitosan and chitosan derivatives. Cationic polysaccharides have, for example, the general formula

G-O-B-N⁺R^{a}R^{b}R^{c} X⁻

G is an anhydroglucose radical, for example starch or cellulose anhydroglucose;

B is a divalent compound group, for example alkylene, oxyalkylene, polyoxyalkylene or hydroxyalkylene;

R^{a}, R^{b} and R^{c}, independently of one another, are alkyl, aryl, alkylaryl, arylalkyl, alkoxyalkyl or alkoxyaryl in each case having up to 18 carbon atoms, where the total number of the carbon atoms in R^{a}, R^{b} and R^{c} is preferably at least 20;

X is a customary counteranion, for example a halogen, acetate, phosphate, nitrate or alkyl sulfate, preferably a chloride.

Cationic celluloses are, for example, those with the INCI names Polyquaternium-10 or Polyquaternium-24. A suitable cationic guar derivative has, for example, the INCI name Guar Hydroxypropyltrimonium Chloride.

Preferred cationic cellulose compounds are those which have at least one quaternary ammonium group, e.g. a copolymer of hydroxyethylcellulose and diallyldimethylammonium chloride (Polyquaternium-4) or the reaction product of hydroxyethylcellulose and an epoxide substituted by a trialkylammonium group (Polyquaternium-10), where the alkyl groups can have from 1 to 20 carbon atoms and are preferably methyl groups. The molecular weight is preferably between 100 000 and 600 000, particularly preferably between 200 000 and 400 000. The nitrogen content is preferably 0.5 to 4%, particularly preferably 1.5 to 3%. A preferred cellulose derivative is Polyquaternium-4, which is sold under the trade names Celquat® H100 and Celquat® L200, of which Celquat® L200 is particularly preferred.

Particularly preferred cation-active substances are chitosan, chitosan salts and chitosan derivatives. The chitosans to be used according to the invention are completely or partially deacetylated chitins. The molecular weight can be spread over a broad spectrum, for example from 20 000 to about 5 million g/mol, e.g. from 30 000 to 70 000 g/mol. Preferably, however, the molecular weight is more than 100 000 g/mol, particularly preferably from 200 000 to 700 000 g/mol. The degree of deacetylation is preferably 10 to 99%, particularly preferably 60 to 99%. A preferred chitosan salt is chitosonium pyrrolidonecarboxylate, e.g. Kytamer® PC with a molecular weight of from about 200 000 to 300 000 g/mol and deacetylation of from 70 to 85%. Suitable chitosan derivatives are quaternized, alkylated or hydroxyalkylated derivatives, e.g. hydroxyethyl-, hydroxypropyl- or hydroxybutylchitosan. The chitosans or chitosan derivatives are preferably in neutralized or partially neutralized form. The degree of neutralization is preferably at least 50%, particularly preferably between 70 and 100%, based on the number of free base groups. Neutralizing agents which can be used are, in principle, all cosmetically compatible inorganic or organic acids, such as, for example, formic acid, tartaric acid, malic acid, lactic acid, citric acid, pyrrolidonecarboxylic acid, hydrochloric acid etc., of which pyrrolidonecarboxylic acid is particularly preferred.

Preferred natural-based cationic polymers:

Cationic cellulose derivatives of hydroxyethylcellulose and diallyldimethylammonium chloride; cationic cellulose derivatives of hydroxyethylcellulose and epoxide substituted by trimethylammonium; chitosan and salts thereof; hydroxyalkylchitosans and salts thereof; alkylhydroxyalkylchitosans and salts thereof; N-hydroxyalkylchitosan alkyl ethers; N-hydroxyalkylchitosan benzyl ethers.

In a further preferred embodiment, the agent according to the invention comprises 0.01 to 15% by weight, preferably 0.5 to 10% by weight, of at least one synthetic or natural nonionic film-forming polymer. Natural polymers are also understood as meaning chemically modified polymers of natural origin. Film-forming polymers are understood as meaning those polymers which, when used in 0.01 to 5% strength aqueous, alcoholic or aqueous-alcoholic solution, are able to deposit a polymer film on the hair.

Suitable synthetic, nonionic film-forming, hair-setting polymers are homopolymers or copolymers which are constructed from at least one of the following monomers: vinylpyrrolidone, vinylcaprolactam, vinyl esters, such as, for example, vinyl acetate, vinyl alcohol, acrylamide, methacrylamide, alkyl- and dialkylacrylamide, alkyl- and dialkylmethacrylamide, alkyl acrylate, alkyl methacrylate, propylene glycol or ethylene glycol, where the alkyl groups of these monomers are preferably C1- to C7-alkyl groups, particularly preferably C1- to C3-alkyl groups. Homopolymers of vinylcaprolactam, of vinylpyrrolidone or of N-vinylformamide, for example are suitable. Further suitable synthetic film-forming, nonionic, hair-setting polymers are, for example, copolymers of vinylpyrrolidone and vinyl acetate, terpolymers of vinylpyrrolidone, vinyl acetate and vinyl propionate, polyacrylamide; polyvinyl alcohols, and polyethylene glycol/polypropylene glycol copolymers. Suitable natural film-forming polymers are, for example, cellulose derivatives, e.g. hydroxyalkylcellulose.

Preferred nonionic polymers are:

Polyvinylpyrrolidone, polyvinylcaprolactam, vinylpyrrolidone/vinyl acetate copolymers, polyvinyl alcohol, isobutylene/ethylmaleimide/hydroxyethylmaleimide copolymer; copolymers of vinylpyrrolidone, vinyl acetate and vinyl propionate.

In one embodiment, the agent according to the invention comprises, as haircare additive, at least one silicone compound in an amount of from preferably 0.01 to 15% by weight, particularly preferably from 0.1 to 5% by weight. The silicone compounds include volatile and nonvolatile silicones and silicones which are insoluble and soluble in the agent. In one embodiment, these are high molecular weight silicones with a viscosity of from 1000 to 2 000 000 cSt at 25° C., preferably 10 000 to 1 800 000 or 100 000 to 1 500 000. The silicone compounds include polyalkyl- and polyarylsiloxanes, in particular with methyl, ethyl, propyl, phenyl, methylphenyl and phenylmethyl groups. Preference is given to polydimethylsiloxanes, polydiethylsiloxanes, polymethylphenylsiloxanes. Preference is also given to shine-imparting, arylated silicones with a refractive index of at least 1.46, or at least 1.52. The silicone compounds include, in particular, the substances with the INCI names Cyclomethicone, Dimethicone, Dimethiconol, Dimethicone Copolyol, Phenyl Trimethicone, Amodimethicone, Trimethylsilylamodimethicone, Stearyl, Siloxysilicate, Polymethylsilsesquioxane, Dimethicone Crosspolymer. Also suitable are silicone resins and silicone elastomers, which are highly crosslinked siloxanes.

Preferred silicones are:

Cyclic dimethylsiloxanes, linear polydimethylsiloxanes, block polymers of polydimethylsiloxane and polyethylene oxide and/or polypropylene oxide, polydimethylsiloxanes with terminal or lateral polyethylene oxide or polypropylene oxide radicals, polydimethylsiloxanes with terminal hydroxyl groups, phenyl-substituted polydimethylsiloxanes, silicone emulsions, silicone elastomers, silicone waxes, silicone gums and amino-substituted silicones.

In one embodiment, the agent according to the invention comprises a photoprotective substance in an amount of from preferably 0.01 to 10% by weight or from 0.1 to 5% by weight, particularly preferably from 0.2 to 2% by weight. The photoprotective substances include, in particular, all of the photoprotective substances specified in EP 1 084 696. Preference is given to: 2-ethylhexyl 4-methoxycinnamate, methyl methoxycinnamate, 2-hydroxy-4-methoxybenzophenone-5-sulfonic acid and polyethoxylated p-aminobenzoates.

In one embodiment, the agent according to the invention comprises at least one hydrophobic oil or wax in an amount of from preferably 0.01 to 20% by weight, particularly preferably from 0.05 to 10% by weight, very particularly preferably from 0.1 to 5% by weight. The liquid, hydrophobic oils have a melting point of less than or equal to 25° C. and a boiling point of preferably above 250° C., in particular above 300° C. In principle, any oil generally known to the person skilled in the art can be used here. Of suitability are vegetable or animal oils, mineral oils (paraffinum liquidum), silicone oils or mixtures thereof. Hydrocarbon oils, e.g. paraffin or isoparaffin oils, squalane, oils from fatty acids and polyols, in particular triglycerides, are suitable. Suitable vegetable oils are, for example, sunflower oil, coconut oil, castor oil, lanolin oil, jojoba oil, corn oil, soybean oil.

In principle, the wax or wax-like substance which can be used is any wax known in the prior art. These include animal, vegetable, mineral and synthetic waxes, microcrystalline waxes, macrocrystalline waxes, solid paraffins, petrolatum, Vaseline, ozokerite, montan wax, Fischer-Tropsch waxes, polyolefin waxes, e.g. polybutene, beeswax, wool wax and derivatives thereof, such as, for example, wool wax alcohols, candelilla wax, olive wax, carnauba wax, Japan wax, apple wax, hydrogenated fats, fatty acid esters and fatty acid glycerides with a solidification point of in each case above 40° C., polyethylene waxes and silicone waxes. The waxes or wax-like substances have a solidification point above 40° C., preferably above 55° C. The needle penetration number (0.1 mm, 100 g, 5 s, 25° C.; according to DIN 51 579) is preferably in the range from 2 to 70, in particular from 3 to 40.

In one embodiment, the agent according to the invention comprises 0.01 to 20% by weight, particularly preferably from 0.05 to 10% by weight, very particularly preferably from 0.1 to 5% by weight, of at least one hair-conditioning additive chosen from betaine; panthenol; panthenyl ethyl ether; sorbitol; protein hydrolysates; plant extracts; A-B block copolymers of alkyl acrylates and alkyl methacrylates; A-B block copolymers of alkyl methacrylates and acrylonitrile; A-B-A block copolymers of lactide and ethylene oxide; A-B-A block copolymers of caprolactone and ethylene oxide; A-B-C block copolymers of alkylene or alkadiene compounds, styrene and alkyl methacrylates; A-B-C block copolymers of acrylic acid, styrene and alkyl methacrylates; star-shaped block copolymers; hyperbranched polymers, dendrimers; intrinsically electrically conductive 3,4-polyethylenedioxythiophenes and intrinsically electrically conductive polyanilines.

In one embodiment, the agent according to the invention comprises 0.01 to 20% by weight, particularly preferably from 0.05 to 10% by weight, very particularly preferably from 0.1 to 5% by weight, of at least one surfactant. The surfactant may be nonionic, anionic, cationic or zwitterionic.

Suitable nonionic surfactants are, for example,
ethoxylated fatty alcohols, fatty acids, fatty acid glycerides or alkylphenols, in particular addition products of from 2 to 30 mol of ethylene oxide and/or 1 to 5 mol of propylene oxide onto C8- to C22-fatty alcohols, onto C12- to C22-fatty acids or onto alkyl phenols having 8 to 15 carbon atoms in the alkyl group,
C12- to C22-fatty acid mono- and diesters of addition products of from 1 to 30 mol of ethylene oxide onto glycerol,
addition products of from 5 to 60 mol of ethylene oxide onto castor oil or onto hydrogenated castor oil,
fatty acid sugar esters, in particular esters of sucrose and one or two C8- to C22-fatty acids, INCI: Sucrose Cocoate, Sucrose Dilaurate, Sucrose Distearate, Sucrose Laurate, Sucrose Myristate, Sucrose Oleate, Sucrose Palmitate, Sucrose Ricinoleate, Sucrose Stearate,
esters of sorbitan and one, two or three C8- to C22-fatty acids and a degree of ethoxylation of from 4 to 20,
polyglyceryl fatty acid esters, in particular of one, two or more C8- to C22-fatty acids and polyglycerol having preferably 2 to 20 glyceryl units,
alkyl glucosides, alkyl oligoglucosides and alkyl polyglucosides having C8 to C22-alkyl groups, e.g. decylglucoside or laurylglucoside.

Suitable anionic surfactants are, for example, salts and esters of carboxylic acids, alkyl ether sulfates and alkyl sulfates, fatty alcohol ester sulfates, sulfonic acid and its salts (e.g. sulfosuccinates or fatty acid isethienates), phosphoric esters and their salts, acylamino acids and their salts. A detailed description of these anionic surfactants is to be found in the publication “FIEDLER—Lexikon der Hilfsstoffe [Lexikon of auxiliaries]”, Volume 1, 5th Edition (2002), pages 97 to 102, to which reference is hereby expressly made. Preferred surfactants are mono-, di- and/or triesters of phosphoric acid whose addition products are from 2 to 30 mol of ethylene oxide onto C8- to C22-fatty alcohols.

Suitable amphoteric surfactants are, for example, derivatives of aliphatic quaternary ammonium, phosphonium and sulfonium compounds of the formula

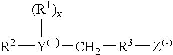

where R1 is a straight-chain or branched-chain alkyl, alkenyl or hydroxyalkyl group having 8 to 18 carbon atoms and 0 to about 10 ethylene oxide units and 0 to 1 glycerol unit; Y is a N-, P- or S-containing group; R2 is an alkyl or monohydroxyalkyl group having 1 to 3 carbon atoms; X is 1 if Y is a sulfur atom and X is 2 if Y is a nitrogen atom or a phosphorus atom; R3 is an alkylene or hydroxyalkylene group having 1 to 4 carbon atoms and Z⁽⁻⁾ is a carboxylate, sulfate, phosphonate or phosphate group.

Other amphoteric surfactants such as betaines are likewise suitable. Examples of betaines includes C8- to C18-alkylbetaines, such as cocodimethylcarboxymethylbetaine, lauryldimethylcarboxymethylbetaine, lauryldimethylalphacarboxyethylbetaine, cetyldimethylcarboxymethylbetaine, oleyldimethylgammacarboxypropylbetaine and laurylbis(2-hydroxypropyl)alphacarboxyethylbetaine; C8- to C18-sulfobetaines, such as cocodimethylsulfopropylbetaine, stearyldimethylsulfopropylbetaine, lauryldimethylsulfoethylbetaine, laurylbis(2-hydroxyethyl)sulfopropylbetaine; the carboxyl derivatives of imidazole, the C8- to C18-alkyldimethylammonium acetates, the C8- to C18-alkyldimethylcarbonylmethylammonium salts, and the C8- to C18-fatty acid alkylamidobetaines, such as, for example, coconut fatty acid amidopropylbetaine and N-coconut fatty acid amidoethyl-N-[2-(carboxymethoxy)ethyl]glycerol (CTFA name: Cocoamphocarboxyglycinate).

Suitable cationic surfactants contain amino groups or quaternized hydrophilic ammonium groups which, in solution, carry a positive charge and can be represented by the general formula

N⁽⁺⁾R¹R²R³R⁴ X⁽⁻⁾

where R1 to R4, independently of one another, are aliphatic groups, aromatic groups, alkoxy groups, polyoxyalkylene groups, alkylamido groups, hydroxyalkyl groups, aryl groups or alkaryl groups having 1 to 22 carbon atoms, where at least one radical has at least 6, preferably at least 8, carbon atoms, and X⁻ is an anion, for example a halogen, acetate, phosphate, nitrate or alkyl sulfate, preferably a chloride. In addition to the carbon atoms and the hydrogen atoms, the aliphatic groups can also contain crosslinkages or other groups, such as, for example, further amino groups.

Examples of suitable cationic surfactants are the chlorides or bromides of alkyldimethylbenzylammonium salts, alkyltrimethylammonium salts, for example cetyltrimethylammonium chloride or bromide, tetradecyltrimethylammonium chloride or bromide, alkyldimethylhydroxyethylammonium chlorides or bromides, the dialkyldimethylammonium chlorides or bromides, alkylpyridinium salts, for example lauryl- or cetylpyridinium chloride, alkylamidoethyltrimethylammonium ether sulfates, and compounds with cationic character, such as amine oxides, for example alkymethylamine oxides or alkylaminoethyldimethylamine oxides. Particular preference is given to C8-22-alkyldimethylbenzylammonium compounds, C8-22-alkyltrimethylammonium compounds, in particular cetyltrimethylammonium chloride, C8-22-alkyldimethylhydroxyethylammonium compounds, di(C8-22-alkyl)dimethylammonium compounds, C8-22-alkylpyridinium salts, C8-22-alkylamidoethyltrimethylammonium ether sulfates, C8-22-alkylmethylamine oxides, C8-22-alkylaminoethyldimethylamine oxides.

In one embodiment, the agent according to the invention comprises 0.01 to 5% by weight, particularly preferably from 0.05 to 1% by weight, of at least one preservative. Suitable preservatives are the substances listed in the International Cosmetic Ingredient Dictionary and Handbook, 9th Edition with the function “preservatives”, e.g. phenoxyethanol, benzyl paraben, butyl paraben, ethyl paraben, isobutyl paraben, isopropyl paraben, methyl paraben, propyl paraben, iodopropynyl butylcarbamate, methyldibromoglutaronitrile, DMDM hydantoin.

In one preferred embodiment, the agent according to the invention comprises at least one pigment. These may be colored pigments which impart color effects to the product mass or to the hair, or they may be luster effect pigments which impart luster effects to the product mass or to the hair. The color or luster effects on the hair are preferably temporary, i.e. they last until the next hair wash and can be removed again by washing the hair with customary shampoos. The pigments are present in the product mass in undissolved form and may be present in an amount of from 0.01 to 25% by weight, particularly preferably from 5 to 15% by weight. The preferred particle size is 1 to 200 μm, in particular 3 to 150 μm, particularly preferably 10 to 100 μm. The pigments are colorants which are virtually insoluble in the application medium, and may be inorganic or organic. Inorganic-organic mixed pigments are also possible. Preference is given to inorganic pigments. The advantage of inorganic pigments is their excellent resistance to light, weather and temperature. The inorganic pigments may be of natural origin, for example produced from chalk, ocher, umber, green earth, burnt sienna or graphite. The pigments may be white pigments, such as, for example, titanium dioxide or zinc oxide, black pigments, such as, for example, iron oxide black, colored pigments, such as, for example, ultramarine or iron oxide red, luster pigments, metal effect pigments, pearlescent pigments, and fluorescent or phosphorescent pigments, where preferably at least one pigment is a colored, nonwhite pigment. Of suitability are metal oxides, hydroxides and oxide hydrates, mixed phase pigments, sulfur-containing silicates, metal sulfides, complex metal cyanides, metal sulfates, chromates and molybdates, and the metals themselves (bronze pigments). Of particular suitability are titanium dioxide (CI 77891), black iron oxide (CI 77499), yellow iron oxide (CI 77492), red and brown iron oxide (CI 77491), manganese violet (CI 77742), ultramarine (sodium aluminum sulfosilicates, CI 77007, Pigment Blue 29), chromium oxide hydrate (CI 77289), Prussian blue (ferric ferrocyanide, CI 77510), carmine (cochineal).

Particular preference is given to pearlescent and colored pigments based on mica which are coated with a metal oxide or a metal oxychloride, such as titanium dioxide or bismuth oxychloride, and optionally further color-imparting substances, such as iron oxides, Prussian blue, ultramarine, carmine etc. and where the color can be determined by varying the layer thickness. Such pigments are sold, for example, under the trade names Rona®, Colorona®, Dichrona® and Timiron® by Merck, Germany.

Organic pigments are, for example, the natural pigments sepia, gamboge, bone charcoal, Cassel brown, indigo, chlorophyll and other plant pigments. Synthetic organic pigments are, for example, azo pigments, anthraquinoids, indigoids, dioxazine, quinacridone, phthalocyanine, isoindolinone, perylene and perinone, metal complex, alkali blue and diketopyrrolopyrrole pigments.

In one embodiment, the agent according to the invention comprises from 0.01 to 10% by weight, particularly preferably from 0.05 to 5% by weight, of at least one particulate substance. Suitable substances are, for example, substances which are solid at room temperature (25° C.) and are in the form of particles. For example, silica, silicates, aluminates, clay earths, mica, salts, in particular inorganic metal salts, metal oxides, e.g. titanium dioxide, minerals and polymer particles are suitable.

The particles are present in the agent in undissolved, preferably stably dispersed form, and, following application to the hair and evaporation of the solvent, can deposit on the hair in solid form. A stable dispersion can be achieved by providing the composition A with a yield point which is large enough to prevent the solid particles from sinking. An adequate yield point can be established using suitable gel formers in a suitable amount.

Preferred particulate substances are silica (silica gel, silicon dioxide) and metal salts, in particular inorganic metal salts, where silica is particularly preferred. Metal salts are, for example, alkali metal or alkaline earth metal halides, such as sodium chloride or potassium chloride; alkali metal or alkaline earth metal sulfates, such as sodium sulfate or magnesium sulfate.

The agent according to the invention is preferably formulated in an aqueous, an alcoholic or in an aqueous-alcoholic medium with preferably at least 10% by weight of water. Alcohols which may be present are, in particular, the lower alcohols having 1 to 4 carbon atoms customarily used for cosmetic purposes, such as, for example, ethanol and isopropanol. The agent according to the invention may be present in a pH range from 2.0 to 9.5. Particular preference is given to the pH range between 4 and 8. Additional cosolvents which may be present are organic solvents or a mixture of solvents with a boiling point below 400° C. in an amount of from 0.1 to 15% by weight, preferably from 1 to 10% by weight. Particularly suitable additional cosolvents are unbranched or branched hydrocarbons, such as pentane, hexane, isopentane and cyclic hydrocarbons, such as cyclopentane and cyclohexane. Further, particularly preferred water-soluble solvents are glycerol, ethylene glycol and propylene glycol in an amount up to 30% by weight.

The agent according to the invention can be used in various application forms, such as, for example, as lotion, as nonaerosol spray lotion, which is used by means of a mechanical device for spraying, as aerosol spray which is sprayed by means of a propellant, as aerosol foam or as nonaerosol foam which is present in combination with a suitable mechanical device for foaming the composition, as hair cream, as hair wax, as gel, as liquid gel, as sprayable gel or as foam gel. A use in the form of a lotion thickened with a customary thickener is also possible.

In one embodiment, the agent according to the invention is in the form of a gel, in the form of a viscous lotion or in the form of a spray gel which is sprayed with a mechanical device and comprises at least one of the abovementioned thickening polymers in an amount of from preferably 0.05 to 10% by weight, particularly preferably from 0.1 to 2% by weight and has a viscosity of at least 250 mPa s (measured using a Bohlin rheometer CS, measurement body C25 at 25° C. and a shear rate of 50 s⁻¹). The viscosity of the gel is preferably from 500 to 50 000 mPa s, particularly preferably from 1000 to 15 000 mPa s at 25° C.

In one embodiment, the agent according to the invention is in the form of an O/W emulsion, a W/O emulsion or a microemulsion, and comprises at least one of the above-mentioned oils or waxes emulsified in water, and at least one of the abovementioned surfactants.

In one embodiment, the agent according to the invention is in the form of a spray product, either in combination with a mechanical pump spray device or in combination with at least one propellant chosen from propane, butane, dimethyl ether and fluorinated hydrocarbons. An aerosol spray additionally comprises preferably 15 to 85% by weight, particularly preferably 25 to 75% by weight, of a propellant and is bottled in a pressurized container. Suitable propellants are, for example, lower alkanes, such as, for example, n-butane, isobutane and propane, or mixtures thereof, and dimethyl ether or fluorinated hydrocarbons, such as F 152a (1,1-difluoroethane) or F 134 (tetrafluoroethane), and also propellants which are in gaseous form at the pressures under consideration, such as, for example, N₂, N₂O and CO₂, and mixtures of the abovementioned propellants.

A nonaerosol hairspray is sprayed with the help of a suitable mechanically operated spraying device. Mechanical spraying devices are understood as meaning those devices which permit the spraying of a composition without use of a propellant. A suitable mechanical spray device used may, for example, be a spray pump or an elastic container provided with a spray valve in which the cosmetic agent according to the invention is bottled under pressure, where the elastic container expands and from which the agent is continuously dispensed as a result of the elastic container contracting upon opening the spray valve.

In one embodiment, the agent according to the invention is in the form of a foamable product (mousse) in combination with a devices for foaming, comprises at least one customary foam-imparting substance known for this purpose, e.g. at least one foam-forming surfactant or at least one foam-forming polymer. Devices for foaming are understood as meaning those devices which enable the foaming of a liquid with or without use of a propellant. A suitable mechanical foaming device which can be used is, for example, a standard commercial pump foamer or an aerosol foaming head. The product is either in combination with a mechanical pump foaming device (pump foam) or in combination with at least one propellant (aerosol foam) in an amount of from preferably 1 to 20% by weight, in particular from 2 to 10% by weight. Propellants are chosen, for example, from propane, butane, dimethyl ether and fluorinated hydrocarbons. The agent is foamed directly prior to application and incorporated into the hair as foam and can then be rinsed out or left in the hair without rinsing.

The foamable products comprise, as active ingredients or additives, preferably polymers which are chosen from chitosan, chitosan salts, chitosan derivatives, cationic cellulose compounds, copolymers of vinylpyrrolidone, vinylcaprolactam and a basic acrylamide monomer or mixtures of these polymers. Suitable chitosan salts, chitosan derivatives, cationic cellulose derivatives are, for example, those mentioned above. Preferred cationic cellulose compounds are copolymers of hydroxyethylcellulose and diallyldimethylammonium chloride (polyquaternium-4) and reaction products of hydroxyethylcellulose and epoxides substituted by a trialkylammonium group (Polyquaternium-10). Preferred chitosan salts are the salts with formic acid, lactic acid and pyrrolidonecarboxylic acid. Preferred copolymers of vinylpyrrolidone, vinylcaprolactam and a basic acrylamide monomer are those in which the acrylamide monomer is dimethylaminopropylacrylamide. Also preferred are foamable products which comprise copolymers of hydroxyethylcellulose and diallyldimethylammonium chloride (polyquaternium-4) and copolymers of vinylpyrrolidone, vinylcaprolactam and dimethylaminopropylacrylamide, and foamable products which comprise copolymers of hydroxyethylcellulose and diallyldimethylammonium chloride (polyquaternium-4) and copolymers of vinylpyrrolidone, vinylcaprolactam and dimethylaminopropylacrylamide, and at least one chitosan salt.

In one embodiment, the agent according to the invention is in the form of a hair wax, i.e. it has a wax-like consistency and comprises at least one of the above-mentioned waxes in an amount of from preferably 0.5 to 30% by weight, and optionally further water-insoluble substances. The wax-like consistency is preferably characterized in that the needle penetration number (unit of measurement 0.1 mm, test weight 100 g, test time 5 s, test temperature 25° C.; according to DIN 51 579) is greater than or equal to 10, particularly preferably greater than or equal to 20 and that the solidification point of the product is preferably greater than or equal to 30° C. and less than or equal to 70° C., particularly preferably in the range from 40 to 55° C. Suitable waxes and water-insoluble substances are, in particular, emulsifiers with an HLB value below 7, silicone oils, silicone waxes, waxes (e.g. wax alcohols, wax acids, wax esters, and in particular natural waxes such as beeswax, carnauba wax, etc.), fatty alcohols, fatty acids, fatty acid esters or hydrophilic waxes, such as, for example, high molecular weight polyethylene glycols with a molecular weight of from 800 to 20 000, preferably from 2000 to 10 000 g/mol.

If the hair-treatment agent according to the invention is in the form of a hair lotion, then it is in the form of an essentially nonviscous or low-viscosity, flowable solution, dispersion or emulsion with a content of at least 10% by weight, preferably 20 to 95% by weight, of a cosmetically compatible alcohol. Alcohols which can be used are, in particular, the lower alcohols having 1 to 4 carbon atoms customarily used for cosmetic purposes, e.g. ethanol and isopropanol.

If the hair-treatment agent according to the invention is in the form of a hair cream, then it is preferably in the form of an emulsion and comprises either additionally viscosity-imparting ingredients in an amount of from 0.1 to 10% by weight, or the required viscosity and creamy consistency is built up in a customary manner through micelle formation with the help of suitable emulsifiers, fatty acids, fatty alcohols, waxes etc.

The examples below serve to illustrate the subject matter of the invention in more detail. Unless stated otherwise, the polymer contents given in each case refer to the solids content.

### EXAMPLES

Substances used in the examples:
Luviset® Clear: Terpolymer of vinylpyrrolidone, methacrylamide and vinylimidazole (BASF)
Surfactant 193: Ethoxylated dimethylpolysiloxane; INCI: PEG-12 Dimethicone (Dow Corning)
Emulgin® L: INCI: PEG-1-PEG-9 LAURYL GLYCOL ETHER
Natrosol G: Hydroxyethylcellulose
Aculyn® 48: PEG-150/STEARYL ALCOHOL/SMDI COPOLYMER, 19% strength in water (Rohm and Haas)
Aquaflex® SF 40: VP/VINYL CAPROLACTAM/DMAPA ACRYLATES COPOLYMER, 40% strength in ethanol (ISP)
Pemulen®: ACRYLATES/C10-30 ALKYL ACRYLATE CROSSPOLYMER
Synthalen® W 2000: ACRYLATES/PALMETH-25 ACRYLATE COPOLYMER (31% strength in water)
Structure® 3001: ACRYLATES/CETETH-20 ITACONATE COPOLYMER 30% strength in water (National Starch)
Dekaben® LMB: IODOPROPYNYL BUTYLCARBAMATE, 10% strength in butylene glycol
Dekaben® LMP: Phenoxyethanol and iodopropynyl butylcarbamate
Celquat® L200: Copolymer of hydroxyethylcellulose and diallyldimethylammonium chloride; Polyquaternium-4
Dow Corning 1401: High molecular weight Dimethiconol, 13% strength in cyclomethicone
Copolymer 845: VP/DIMETHYLAMINOETHYLMETHACRYLATE COPOLYMER, 20% strength in water (ISP)
Abilquat® 3270: Quaternium-80, 50% strength in propylene glycol (Goldschmidt)
Aquaflex® FX-64: ISOBUTYLENE/ETHYLMALEIMIDE/HYDROXY-ETHYLMALEIMIDE COPOLYMER, 40% strength in water/ethanol (ISP)
Diaformer Z-711: ACRYLATES/LAURYL ACRYLATE/STEARYL ACRYLATE/ETHYLAMINE OXIDE METHACRYLATE COPOLYMER, 40% strength (Clariant)

### Example 1

### Liquid Gel

| | |
|---|---|
| Luviset ® Clear | 1.0 g |
| Surfactant 193 | 1.5 g |
| Carbomer | 0.3 g |
| AMP 95% | 0.3 g |
| Emulgin L | 0.2 g |
| Perfume | 0.15 g |
| Natrosol ® | 0.4 g |
| Ethanol | 16.5 g |
| Water | ad 100 |

### Example 2

### Liquid Gel

| | |
|---|---|
| Luviset ® Clear | 1.0 g |
| Vinylpyrrolidone/vinyl acetate copolymer | 1.0 g |
| Surfactant 193 | 1.0 g |
| Xanthan Gum | 1.2 g |
| Citric acid | 0.1 g |
| Perfume | 0.15 g |
| Ethanol | 6.5 g |
| DMDM Hydantoin | 0.3 g |
| Water | ad 100 |

### Example 3

### Liquid Gel

| | |
|---|---|
| Luviset ® Clear | 0.5 g |
| Glucose | 7.0 g |
| Propylene glycol | 3.8 g |
| Hydroxypropylguar | 0.3 g |
| AMP 95% | 0.2 g |
| PEG-25 PABA | 0.5 g |
| PEG-40 Hydrogenated Castor Oil | 0.18 g |
| PPG-1-PEG-9 Lauryl Glycol Ether | 0.18 g |
| Perfume | 0.15 g |
| Ethanol | 16.5 g |
| Water | ad 100 |

### Example 4

### Spray Gel

| | |
|---|---|
| Luviset ® Clear | 1.5 g |
| VP/VA COPOLYMER (Luviskol ® VA 64) | 3.0 g |
| Ethanol | 18 g |
| Aminomethylpropanol 95% | 0.1 g |
| PEG-40 Hydrogenated Castor Oil | 0.2 g |
| Perfume | 0.2 g |
| Aculyn ® 48 | 0.5 g |
| Water | ad 70 |

The composition is packaged in a packaging with pump spray device.

### Example 5

### Rapidly Drying Gel

| | |
|---|---|
| Luviset ® Clear | 3.0 g |
| Aquaflex ® SF 40 | 2.8 g |
| Surfactant 193 | 1.5 g |
| Pemulen ® | 0.35 g |
| AMP 95% | 0.26 g |
| Methylmethoxycinnamate | 0.30 g |
| Perfume | 0.30 g |
| Ethanol 510 | 34.2 g |
| Water | ad 100 |

### Example 6

### Gel Spray

| | |
|---|---|
| Luviset ® Clear | 1.0 g |
| Surfactant 193 | 1.0 g |
| Carbomer (Carbopol) | 0.23 g |
| AMP 95% | 0.22 g |
| Perfume | 0.15 g |
| Ethanol 510 | 6.5 g |
| Water | ad 100 |

The composition is packaged in a packaging with pump spray device.

### Example 7

### Blow-Drying Gel

| | |
|---|---|
| Luviset ® Clear | 1.0 g |
| Surfactant 193 | 1.0 g |
| Hydroxypropylcellulose (Klucel ® HF) | 0.95 g |
| Citric acid | 0.1 g |
| Perfume | 0.15 g |
| Ethanol 510 | 6.5 g |
| Water | ad 100 |

### Example 8

### Gel

| | |
|---|---|
| Luviset ® Clear | 1.0 g |
| Polyvinylpyrrolidone K 90 | 1.8 g |
| Surfactant 193 | 1.5 g |
| Synthalen ® W 2000 | 1.0 g |
| AMP 95% | 0.3 g |
| PEG-25 PABA (Uvinul ® P 25) | 0.3 g |
| Panthenol | 0.15 g |
| Perfume | 0.3 g |
| Ethanol 510 | 34.2 g |
| Keratin hydrolysate | 0.1 g |
| Water | ad 100 |

### Example 9

### Gel—Strong Hold

| | |
|---|---|
| Luviset ® Clear | 1.0 g |
| VA/CROTONATES COPOLYMER (Luviset ® CA 66) | 2.5 g |
| Sorbitol | 4.2 g |
| Carbomer (Tego Carbomer) | 0.8 g |
| AMP 95% | 0.3 g |
| Methylparaben | 0.2 g |
| PEG-40 Hydrogenated Castor Oil | 0.2 g |
| Panthenol | 0.1 g |
| Perfume | 0.2 g |
| Ethanol 510 | 5.0 g |
| Water | ad 100 |

### Example 10

### Gel—Strong Hold

| | |
|---|---|
| Luviset ® Clear | 1.0 g |
| Aquaflex ® SF 40 | 1.5 g |
| Vinyl acetate/crotonic acid copolymer | 1.2 g |
| Sorbitol | 4.2 g |
| Structure ® 3001 | 0.12 g |
| AMP 95% | 0.35 g |
| PEG-25 PABA | 0.5 g |
| Dekaben ® LMB | 0.2 g |
| PEG-40 Hydrogenated Castor Oil | 0.2 g |
| Panthenol | 0.1 g |
| Perfume | 0.2 g |
| Ethanol 510 | 5.0 g |
| Water | ad 100 |

### Example 11

### Gel—Normal Hold

| | |
|---|---|
| Luviset ® Clear | 1.5 g |
| Glycerol | 5.2 g |
| Propylene glycol | 4.0 g |
| Ammonium Acryloyldimethyltaurate/VP | 0.35 g |
| Copolymer (Aristoflex AVC) | |
| AMP 95% | 0.26 g |
| Polysorbate-40 | 1.0 g |
| Methylparaben | 0.2 g |
| PEG-25 PABA | 0.5 g |
| Perfume | 0.2 g |
| Ethanol 510 | 4.5 g |
| Water | ad 100 |

### Example 12

### Pump—Setting Foam

| | |
|---|---|
| Luviset ® Clear | 1.3 |
| Vinyl acetate/crotonic acid copolymer | 0.3 |
| Cocamidopropyl Hydroxysultaine | 0.4 |
| Citric acid | 0.1 |
| Ethanol 510 | 8.9 |
| Betaine | 0.1 |
| Perfume | 0.15 |
| Water | ad 100 |

The composition is packaged in a packaging with mechanically operated pump foaming device.

### Example 13

### Pump—Setting Foam

| | |
|---|---|
| Luviset ® Clear | 1.5 |
| Acrylic acid/ethyl acrylate/N-tert- | 0.4 |
| butylacrylamide Copolymer | |
| Cocamidopropyl Hydroxysultaine | 0.4 |
| Citric acid | 0.1 |
| Dekaben ® LMP | 0.2 |
| Camomile blossom extract | 0.1 |
| Perfume | 0.15 |
| Water | ad 100 |

The composition is packaged in a packaging with mechanically operated pump foaming device.

### Example 14

### Pump—Setting Foam

| | |
|---|---|
| Luviset ® Clear | 1.2 |
| Polyquaternium- 6 | 0.35 |
| Cocamidopropyl Hydroxysultaine | 0.4 |
| Panthenol | 0.1 |
| Ethanol 510 | 8.9 |
| Betaine | 0.1 |
| Perfume | 0.15 |
| Water | ad 100 |

The composition is packaged in a packaging with mechanically operated pump foaming device.

### Example 15

### Pump—Setting Foam

| | |
|---|---|
| Luviset ® Clear | 2.5 g |
| Ethanol | 8.9 g |
| Cocamidopropyl Hydroxysultaine | 0.2 g |
| Cetyltrimethylammonium chloride | 0.2 g |
| Perfume | 0.15 g |
| Silk fibroin hydrolysate (Silkpro ®) | 0.1 g |
| Water | ad 100 |

The composition is packaged in a packaging with mechanically operated pump foaming device.

### Example 16

### Pump—Setting Foam

| | |
|---|---|
| Luviset ® Clear | 2.0 g |
| Celquat ® L200 | 0.3 g |
| Ethanol | 8.9 g |
| Cocamidopropyl Hydroxysultaine | 0.2 g |
| Cetyltrimethylammonium chloride | 0.2 g |
| Perfume | 0.15 g |
| Citric acid | 0.1 g |
| Betaine | 0.1 g |
| Water | ad 100 |

The composition is packaged in a packaging with mechanically operated pump foaming device.

### Example 17

### Pump—Setting Foam

| | |
|---|---|
| Luviset ® Clear | 1.3 |
| Polyquaternium-11 | 0.3 |
| Cocamidopropyl Hydroxysultaine | 0.4 |
| Propylene glycol | 1.0 |
| Methylparaben | 0.2 |
| Perfume | 0.15 |
| Water | ad 100 |

The composition is packaged in a packaging with mechanically operated pump foaming device.

### Example 18

### Pump—Setting Foam

| | |
|---|---|
| Luviset ® Clear | 1.8 |
| Cocamidopropyl Hydroxysultaine | 0.4 |
| Rosemary leaf extract (Extrapon ® Rosemary) | 0.1 |
| Ethanol 510 | 8.9 |
| Extrapon ® seven herbs - extract | 0.1 |
| Panthenyl ethyl ether | 0.1 |
| Perfume | 0.15 |
| Water | ad 100 |

The composition is packaged in a packaging with mechanically operated pump foaming device.

### Example 19

### Aerosol—Setting Foam—Normal Hold

| | |
|---|---|
| Luviset ® Clear | 1.5 |
| Butyl monoester of methyl vinyl | 0.5 |
| ether/maleic acid copolymer | |
| Butane | 4.0 |
| Propane | 4.0 |
| Ethanol 510 | 8.9 |
| PEG-25 PABA | 0.4 |
| Betaine | 0.15 |
| Perfume | 0.15 |
| Laureth-4 | 0.2 |
| Cetrimonium bromide | 0.05 |
| Amodimethicone | 0.5 |
| Water | ad 100 |

The composition is bottled in an aerosol can with foaming head.

### Example 20

### Aerosol—Setting Foam—Normal Hold

| | |
|---|---|
| Luviset ® Clear | 1.5 |
| Polyquaternium-47 | 0.5 |
| Butane | 4.0 |
| Propane | 4.0 |
| Betaine | 0.15 |
| Dow Corning 1401 | 0.25 |
| 2-Ethylhexyl 4-methoxycinnamate | 0.2 |
| Perfume | 0.15 |
| Laureth-4 | 0.2 |
| Cetrimonium chloride | 0.07 |
| Water | ad 100 |

The composition is bottled in an aerosol can with foaming head.

### Example 21

### Aerosol—Setting Foam—Extra Strong Hold

| | |
|---|---|
| Luviset ® Clear | 2.1 |
| Copolymer 845 | 2.5 |
| Polyquaternium-4 | 1.0 |
| Butane | 4.0 |
| Propane | 4.0 |
| Panthenol | 0.2 |
| Perfume | 0.2 |
| Abilquat ® 3270 | 0.7 |
| Cetrimonium chloride | 0.07 |
| Water | ad 100 |

The composition is bottled in an aerosol can with foaming head.

### Example 22

### Aerosol—Setting Foam—Extra Strong Hold

| | |
|---|---|
| Luviset ® Clear | 2.1 |
| Vinyl acetate/crotonic acid copolymer | 0.6 |
| Polyquaternium-7 | 0.5 |
| Butane | 4.0 |
| Propane | 4.0 |
| Ethanol 510 | 8.9 |
| PEG-25 PABA | 0.4 |
| Panthenol | 0.2 |
| Perfume | 0.2 |
| Laureth-4 | 0.2 |
| C9-C11 Pareth-8 | 0.07 |
| Water | ad 100 |

The composition is bottled in an aerosol can with foaming head.

### Example 23

### Setting Spray

| | |
|---|---|
| Luviset ® Clear | 1.5 |
| Aquaflex ® FX-64 | 1.0 |
| Ethanol 510 | 2.7 |
| Polyquaternium-35 | 1.0 |
| PEG-25 PABA | 0.7 |
| Panthenol | 0.35 |
| Perfume | 0.25 |
| Cetrimonium chloride | 0.2 |
| PEG-40 Hydrogenated Castor Oil | 0.21 |
| Water | ad 100 |

The composition is bottled in a packaging with pump spray device.

### Example 24

### Setting Spray

| | |
|---|---|
| Luviset ® Clear | 2.5 g |
| Octylacrylamide/Acrylates/Butylaminoethyl- | 2.0 g |
| methacrylate Copolymer (Amphomer ®) | |
| Ethanol | 28.5 g |
| Aminomethylpropanol 95% | 0.6 g |
| Perfume | 0.25 g |
| Cetyltrimethylammonium bromide | 0.20 g |
| Water | ad 60 |

The composition is bottled in a packaging with pump spray device.

### Example 25

### Setting Spray

| | |
|---|---|
| Luviset ® Clear | 1.0 g |
| Octylacrylamide/Acrylates/Butylaminoethyl- | 0.65 g |
| methacrylate Copolymer (Amphomer ®) | |
| Celquat ® L200 | 0.2 g |
| Ethanol | 28.5 g |
| Aminomethylpropanol 95% | 0.6 g |
| Perfume | 0.25 g |
| Cetyltrimethylammonium chloride | 0.20 g |
| Water | ad 60 |

The composition is bottled in a packaging with pump spray device.

### Example 26

### Nonaerosol Blow-Drying Lotion

| | |
|---|---|
| Luviset ® Clear | 2.8 g |
| Vinyl Caprolactam/VP/Dimethylaminoethyl | 2.0 g |
| Methacrylate Copolymer (Advantage ® S) | |
| Ethanol | 28.5 g |
| Perfume | 0.25 g |
| Cetyltrimethylammonium chloride | 0.20 g |
| Water | ad 60 |

### Example 27

### Nonaerosol Blow-Drying Lotion

| | |
|---|---|
| Luviset ® Clear | 3.1 g |
| Celquat ® L200 | 0.05 g |
| Diaformer ® Z-711 | 0.5 g |
| Ethanol | 27 g |
| Betaine | 0.1 g |
| Perfume | 0.25 g |
| PEG-40 Hydrogenated Castor Oil | 0.21 g |
| Cetyltrimethylammonium bromide | 0.20 g |
| Water | ad 100 |

### Example 28

### Nonaerosol Blow-Drying Lotion

| | |
|---|---|
| Luviset ® Clear | 3.00 g |
| Sodium polystyrenesulfonate (Flexan ®) | 2.30 g |
| Perfume | 0.20 g |
| Phenyltrimethicone (Baysilon ® oil PD 5) | 0.02 g |
| Water | 10.00 g |
| Ethanol | ad 100 |

The active ingredient solution is bottled in the ratio 45:55 with DME as propellant in an aerosol can.

### Example 29

### VOC 80 Pump Spray—Strong Hold

| | |
|---|---|
| Luviset ® Clear | 6.5 |
| t-Butyl acrylate/Ethyl acrylate/Methacrylic | 0.5 |
| acid Copolymer (Luvimer ® 100 P) | |
| Perfume | 0.2 |
| AMP | 0.10 |
| Betaine | 0.05 |
| Ethanol 510 | 55 |
| Demineralized water | ad 100 |

The composition is bottled in a packaging with pump spray device.

### Example 30

### Aerosol—Hairspray

| | |
|---|---|
| Octylacrylamide/Acrylic acid/Butylaminoethyl | 3.00 |
| methacrylate/Methyl methacrylate/ | |
| hydroxypropyl methacrylate Copolymer | |
| (Amphomer ®) | |
| Luviset ® Clear | 1.50 |
| Phenyl trimethicone (Baysilon ® oil PD 5) | 0.02 |
| Perfume | 0.20 |
| Water | 10.00 |
| AMP 95% | 0.48 |
| Ethanol 510 | ad 100 |

The active ingredient solution is bottled in the ratio 45:55 with DME as propellant in an aerosol can.

### Example 31

### Aerosol—Hairspray

| | |
|---|---|
| t-Butyl acrylate/Ethyl acrylate/Methacrylic | 3.3 |
| acid Copolymer (Luvimer ® 100 P) | |
| Luviset ® Clear | 3.3 |
| VA/CROTONATES COPOLYMER | 1.0 |
| (Luviset ® CA 66) | |
| Perfume | 0.2 |
| Water | 10.0 |
| AMP 95% | 0.84 |
| Ethanol 510 | ad 100 |

The active ingredient solution is bottled in the ratio 45:55 with DME as propellant in an aerosol can.

### Example 32

### Aerosol—Hairspray

| | |
|---|---|
| Luviset ® Clear | 2.50 g |
| t-Butyl acrylate/Ethyl acrylate/Methacrylic | 3.30 g |
| acid Copolymer (Luvimer ® 100 P) | |
| Aminomethylpropanol 95% | 0.85 g |
| Perfume | 0.20 g |
| Baysilon ® oil PD 5 | 0.02 g |
| Water | 10.00 g |
| Ethanol | ad 100 |

The active ingredient solution is bottled in the ratio 45:55 with DME as propellant in an aerosol can.

### Example 33

### Volumizing Aerosol Foam

| | |
|---|---|
| VP/Methacrylamide/Vinyl Imidazole Copolymer | 1.9 |
| (Luviset ® Clear) | |
| Polyquaternium-4 (Celquat ® L200) | 0.9 |
| VP/Vinyl Caprolactam/DMAPA Acrylates | 0.4 |
| Copolymer (Aquaflex ® SF 40) | |
| Laureth-4 | 0.2 |
| Cetrimonium chloride | 0.1 |
| Perfume | 0.1 |
| Butane | 2.2 |
| Propane | 3 |
| Isobutane | 0.8 |
| Water | ad 100 |

The composition is bottled in an aerosol can with foaming head. Through use of the product on the hair, the hairstyle is given long-lasting volume.

### Example 34

### Volumizing Aerosol Foam

| | |
|---|---|
| VP/Methacrylamide/Vinyl Imidazole Copolymer | 1.1 |
| (Luviset ® Clear) | |
| Chitosan | 1.0 |
| Polyquaternium-4 (Celquat ® L200) | 0.9 |
| VP/Vinyl Caprolactam/DMAPA Acrylates | 0.4 |
| Copolymer (Aquaflex ® SF 40) | |
| Pyrrolidone carboxylic acid | 0.85 |
| Laureth-4 | 0.2 |
| Cetrimonium chloride | 0.1 |
| Perfume | 0.1 |
| Butane | 2.2 |
| Propane | 3 |
| Isobutane | 0.8 |
| Water | ad 100 |

The composition is bottled in an aerosol can with foaming head. Through use of the product on the hair, the hairstyle is given long-lasting volume.

### Example 35

### Volumizing Aerosol Foam

| | |
|---|---|
| VP/Methacrylamide/Vinyl Imidazole Copolymer | 2.0 |
| (Luviset ® Clear) | |
| Chitosan | 0.27 |
| Polyquaternium-4 (Celquat ® L200) | 1.0 |
| Pyrrolidone carboxylic acid | 0.23 |
| Laureth-4 | 0.2 |
| Cetrimonium chloride | 0.1 |
| Perfume, preservative | q.s. |
| Water | ad 100 |

The composition is bottled with propane/butane 4.8 bar in the ratio of active ingredient solution:propellant gas=94:6 in an aerosol can with foaming head. Through use of the product on the hair, the hairstyle is given long-lasting volume.

1. A hair-treatment agent with a content of a combination of
(A) a terpolymer constructed from vinylpyrrolidone, methacrylamide and vinylimidazole and
(B) at least one active ingredient or additive chosen from viscosity-modifying substances, haircare substances, hair-setting substances, silicone compounds, photoprotective substances, oils, waxes, preservatives, pigments, soluble dyes, particulate substances, and surfactants
in a suitable cosmetic base.

2. The agent as claimed in claim 1, characterized in that the terpolymer (A) is present in an amount of from 0.1 to 10% by weight and that the viscosity-modifying substance present in an amount of from 0.1 to 5% by weight is, a thickening polymer, chosen from copolymers of at least one first monomer type, which is chosen from acrylic acid and methacrylic acid, and at least one second monomer type, which is chosen from esters of acrylic acid and ethoxylated fatty alcohol; crosslinked polyacrylic acid; crosslinked copolymers of at least one first monomer type, which is chosen from acrylic acid and methacrylic acid, and at least one second monomer type, which is chosen from esters of acrylic acid with C10- to C30-alcohols; copolymers of at least one first monomer type, which is chosen from acrylic acid and methacrylic acid, and at least one second monomer type, which is chosen from esters of itaconic acid and ethoxylated fatty alcohol; copolymers of at least one first monomer type, which is chosen from acrylic acid and methacrylic acid, at least one second monomer type, which is chosen from esters of itaconic acid and ethoxylated C10- to C30- alcohol and a third monomer type, chosen from C1- to C4-aminoalkyl acrylates; copolymers of two or more monomers chosen from acrylic acid, methacrylic acid, acrylic esters and methacrylic esters; copolymers of vinylpyrrolidone and ammonium acryloyldimethyltaurate; copolymers of ammonium acryloyldimethyltaurate and monomers chosen from esters of methacrylic acid and ethoxylated fatty alcohols; hydroxyethylcellulose; hydroxypropylcellulose; hydroxypropylguar; glyceryl polyacrylate; glyceryl polymethacrylate; copolymers of at least one C2-, C3- or C4-alkylene and styrene; polyurethanes; hydroxypropyl starch phosphate; polyacrylamide; copolymer of maleic anhydride and methyl vinyl ether crosslinked with decadiene; carob seed flour; guar gum; xanthan; dehydroxanthan; carrageenan; karaya gum; hydrolyzed corn starch; copolymers of polyethylene oxide, fatty alcohols and saturated methylenediphenyl diisocyanate.

3. The agent as claimed in claim 1 characterized in that the haircare or hair-setting substance present is a polymer with anionic or anionizable groups, chosen from terpolymers of acrylic acid, ethyl acrylate and N-tert-butylacrylamide; crosslinked or uncrosslinked vinyl acetate/crotonic acid copolymers; terpolymers of tert-butyl acrylate, ethyl acrylate and methacrylic acid; sodium polystyrenesulfonate; copolymers of vinyl acetate, crotonic acid and vinyl propionate; copolymers of vinyl acetate, crotonic acid and vinyl neodecanoate; aminomethylpropanol acrylate copolymers; copolymers of vinylpyrrolidone and at least one further monomer chosen from acrylic acid, methacrylic acid, acrylic esters and methacrylic esters; copolymers of methyl vinyl ether and maleic monoalkyl esters; aminomethylpropanol salts of copolymers of allyl methacrylate and at least one further monomer chosen from acrylic acid, methacrylic acid, acrylic esters and methacrylic esters; crosslinked copolymers of ethyl acrylate and methacrylic acid; copolymers of vinyl acetate, mono-n-butyl maleate and isobornyl acrylate; copolymers of two or more monomers chosen from acrylic acid, methacrylic acid, acrylic esters and methacrylic esters, copolymers of octylacrylamide and at least one monomer chosen from acrylic acid, methacrylic acid, acrylic esters and methacrylic esters; polyesters of diglycol, cyclohexanedimethanol, isophthalic acid and sulfoisophthalic acid.

4. The agent as claimed in claim 1 characterized in that the haircare or hair-setting substance present is a polymer with cationic or cationizable groups chosen from cationic cellulose derivatives of hydroxyethylcellulose and diallyldimethylammonium chloride; cationic cellulose derivatives of hydroxyethylcellulose and with trimethylammonium substituted epoxide; poly(dimethyldiallylammonium chloride); copolymers of acrylamide and dimethyldiallylammonium chloride; quaternary ammonium polymers formed by the reaction of diethyl sulfate and a copolymer of vinylpyrrolidone and dimethylaminoethyl methacrylate; quaternary ammonium polymers of methylvinylimidazolium chloride and vinylpyrrolidone; polyquaternium-35; polymer of trimethylammonium-ethyl methacrylate chloride; polyquaternium-57; dimethylpolysiloxanes terminally substituted by quaternary ammonium groups; copolymer of vinylpyrrolidone, dimethylaminopropylmethacrylamide and methacryloylaminopropyllauryldimethylammonium chloride; chitosan and salts thereof; hydroxyalkylchitosans and salts thereof; alkylhydroxyalkylchitosans and salts thereof; N-hydroxyalkylchitosan alkyl ethers; N-hydroxyalkylchitosan benzyl ethers; copolymer of vinylcaprolactam, vinylpyrrolidone and dimethylaminoethyl methacrylate; copolymers of vinylpyrrolidone and dimethylaminoethyl methacrylate, copolymers of vinylpyrrolidone, vinylcaprolactam and dimethylaminopropylacrylamide; polyesters or oligoesters constructed from at least one first monomer type which is chosen from hydroxy acid substituted by at least one quaternary ammonium group.

5. The agent as claimed in claim 1 characterized in that the haircare or hair-setting substance present is a zwitterionic and/or an amphoteric polymer chosen from copolymers of octylacrylamide, acrylic acid, butylaminoethyl methacrylate, methyl methacrylate and hydroxypropyl methacrylate; copolymers of lauryl acrylate, stearyl acrylate, ethylamine oxide methacrylate and at least one monomer chosen from acrylic acid, methacrylic acid, acrylic esters and methacrylic esters; copolymers of methacryloylethylbetaine and at least one monomer chosen from methacrylic acid and methacrylic esters; copolymers of acrylic acid, methyl acrylate and methacrylamidopropyltrimethylammonium chloride; oligomers or polymers preparable from quaternary croton betaines or quaternary croton betaine esters.

6. The agent as claimed in claim 1 characterized in that the haircare or hair-setting substance present is a nonionic polymer chosen from polyvinylpyrrolidone, polyvinylcaprolactam, vinylpyrrolidone/vinyl acetate copolymers, polyvinyl alcohol, isobutylene/ethylmaleimide/hydroxyethylmaleimide copolymer; copolymers of vinylpyrrolidone, vinyl acetate and vinyl propionate.

7. The agent as claimed in claim 1 characterized in that at least one silicone compound is present chosen from cyclic dimethylsiloxanes, linear polydimethylsiloxanes, block polymers of polydimethylsiloxane and polyethylene oxide and/or polypropylene oxide, polydimethylsiloxanes with terminal or lateral polyethylene oxide or polypropylene oxide radicals, polydimethylsiloxanes with terminal hydroxyl groups, phenyl-substituted polydimethylsiloxanes, silicone emulsions, silicone elastomers, silicone waxes, silicone gums and amino-substituted silicones.

8. The agent as claimed in claim 1 characterized in that at least one photo-protective substance is present chosen from 2-ethylhexyl 4-methoxycinnamate, methyl methoxycinnamate, 2-hydroxy-4-methoxybenzophenone-5-sulfonic acid and polyethoxylated p-aminobenzoates.

9. The agent as claimed in claim 1 characterized in that at least one oil or wax is present chosen from paraffin waxes, polyolefin waxes, wool wax, wool wax alcohols, candelilla wax, olive wax, carnauba wax, Japan wax, apple wax, hydrogenated fats, fatty acid esters, fatty acid glycerides, fatty acid triglycerides, mineral oils, isoparaffin oils, paraffin oils, squalane, sunflower oil, coconut oil, castor oil, lanolin oil, jojoba oil, corn oil, soybean oil.

10. The agent as claimed in claim 1 characterized in that at least one haircare or hair-setting substance is present chosen from betaine; panthenol; panthenyl ethyl ether; sorbitol; protein hydrolysates; plant extracts; A-B block copolymers of alkyl acrylates and alkyl methacrylates; A-B block copolymers of alkyl methacrylates and acrylonitrile; A-B-A block copolymers of lactide and ethylene oxide; A-B-A block copolymers of caprolactone and ethylene oxide; A-B-C block copolymers of alkylene or alkadiene compounds, styrene and alkyl methacrylates; A-B-C block copolymers of acrylic acid, styrene and alkyl methacrylates; star-shaped block copolymers; hyperbranched polymers, dendrimers, intrinsically electrically conductive 3,4-polyethylenedioxythiophenes and intrinsically electrically conductive polyanilines.

11. The agent as claimed in claim 1 characterized in that at least one surfactant is present chosen from addition products of from 2 to 30 mol of ethylene oxide and/or 1 to 5 mol of propylene oxide onto C8- to C22-fatty alcohols, addition products of from 2 to 30 mol of ethylene oxide and/or 1 to 5 mol of propylene oxide onto C12- to C22-fatty acids, addition products of from 2 to 30 mol of ethylene oxide and/or 1 to 5 mol of propylene oxide onto alkyl phenols having 8 to 15 carbon atoms in the alkyl group, C12- to C22-fatty acid mono- and diesters of addition products of from 1 to 30 mol of ethylene oxide onto glycerol, addition products of from 5 to 60 mol of ethylene oxide onto castor oil or onto hydrogenated castor oil, mono-, di- or triesters of phosphoric acid with addition products of from 2 to 30 mol of ethylene oxide onto C8- to C22-fatty alcohols, esters of sucrose and one or two C8- to C22-fatty acids, esters of sorbitan and one, two or three C8- to C22-fatty acids and a degree of ethoxylation of from 4 to 20, polyglyceryl fatty acid esters of one, two or more C8- to C22-fatty acids and polyglycerol having 2 to 20 glyceryl units, alkyl glycosides, C8-22-alkyldimethylbenzylammonium compounds, C8-22-alkyltrimethylammonium compounds, C8-22-alkyldimethylhydroxyethylammonium compounds, di(C8-22-alkyl)dimethylammonium compounds, C8-22-alkylpyridinium salts, C8-22-alkylamidoethyltrimethylammonium ether sulfates, C8-22-alkylmethylamine oxides, C8-22-alkylaminoethyldimethylamine oxides.

12. The agent as claimed in claim 1 characterized in that a preservative is present chosen from phenoxyethanol, benzyl paraben, butyl paraben, ethyl paraben, isobutyl paraben, isopropyl paraben, methyl paraben, propyl paraben, methyldibromoglutaronitrile, DMDM hydantoin.

13. The agent as claimed in claim 1 characterized in that at least one pigment is present chosen from titanium dioxide (CI 77891), black iron oxide (CI 77499), yellow iron oxide (CI 77492), red and brown iron oxide (CI 77491), manganese violet (CI 77742), ultramarine (CI 77007), chromium oxide hydrate (CI 77289), Prussian blue (CI 77510), bismuth oxychloride (CI 77163), carmine (cochineal), pearlescent and colored pigments based on mica which are coated with a metal oxide or a metal oxychloride, such as titanium dioxide or bismuth oxychloride, and optionally further color-imparting substances, such as iron oxides, iron blue, ultramarine or carmine and where the color is determined by varying the layer thickness.

14. The agent as claimed in claim 1 characterized in that at least one particulate substance is present chosen from silica, silicates, aluminates, clay earths, mica, insoluble metal salts, metal oxides, minerals and insoluble polymer particles.

15. The agent as claimed in claim 1 characterized in that it is in the form of a gel, in the form of a viscous lotion or in the form of a spray gel which is sprayed with a mechanical device, comprises at least one thickening polymer according to claim 2 and has a viscosity of at least 250 mPa s (measured using a Bohlin rheometer CS, measurement body C25 at 25° C. and a shear rate of 50 s⁻¹).

16. The agent as claimed in claim 1 characterized in that it is in the form of an O/W emulsion, a W/O emulsion or a microemulsion, and comprises at least one oil or wax according to claim 9, and at least one surfactant according to claim 11.

17. The agent as claimed in claim 1 characterized in that it is in the form of a spray product and is either in combination with a mechanical pump spray device or in combination with at least one propellant chosen from propane, butane, dimethyl ether and fluorinated hydrocarbons.

18. The agent as claimed in claim 1 characterized in that it is in the form of a foamable product in combination with a device for foaming, comprises at least one foam-forming surfactant or at least one foam-forming polymer and is either in combination with a mechanical pump foam device or in combination with at least one propellant chosen from propane, butane, dimethyl ether and fluorinated hydrocarbons.

19. The agent as claimed in claim 1 characterized in that it is in the form of a hair wax.

20. The agent as claimed in claim 1 characterized in that it is in the form of a foamable product in combination with a device for foaming, comprises at least one foam-forming surfactant or at least one foam-foaming polymer and is either in combination with a mechanical pump foaming device or in combination with at least one propellant, and the active ingredient or additive is chosen from chitosan, chitosan salts, chitosan derivatives, cationic cellulose compounds, copolymers of vinylpyrrolidone, vinylcaprolactam and a basic acrylamide monomer or mixtures of these polymers.

21. The agent as claimed in claim 20, characterized in that the active ingredient or additive is chosen from chitosan, chitosan salts, copolymers of hydroxyethylcellulose and diallyldimethylammonium chloride (polyquaternium-4), copolymers of vinylpyrrolidone, vinylcaprolactam and dimethylaminopropylacrylamide or mixtures of these polymers.

22. The agent as claimed in claim 20 characterized in that either copolymers of hydroxyethylcellulose and diallyldimethylammonium chloride (polyquaternium-4) or copolymers of vinylpyrrolidone, vinylcaprolactam and dimethylaminopropylacrylamide are present.

23. The agent as claimed in claim 20 characterized in that either copolymers of hydroxyethylcellulose and diallyldimethylammonium chloride (polyquaternium-4), or copolymers of vinylpyrrolidone, vinylcaprolactam and dimethylaminopropylacrylamide, or at least one chitosan salt are present.
